(19) United States
(12) Patent Application Publication
(10) Pub. No.: US 2024/0277534 A1
(43) Pub. Date: Aug. 22, 2024

(54) BANDAGE APPLICATOR

(71) Applicant: ALMA PLUS LLC, Woodmere, NY (US)

(72) Inventors: Alon RAZGOUR, Tel Aviv-Yafo (IL); Shachar BAREL, Woodmere, NY (US)

(21) Appl. No.: 18/559,937

(22) PCT Filed: May 9, 2022
(86) PCT No.: PCT/IL2022/050479

§ 371 Date: Nov. 9, 2023

Related U.S. Application Data
(60) Provisional application No. 63/186,228, filed on May 10, 2021.

(51) Int. Cl. A61F 15/00; A61F 13/00

(52) U.S. Cl. CPC A61F 15/005; A61F 13/00063

(57) ABSTRACT

The invention relates to a two-state roll-bandage applicator, comprising (a) an elongated cylinder configured for insertion into a central lumen of a roll bandage, the cylinder comprising one or more flanges configured to retain the cylinder within the lumen of said roll bandage; (b) a handle configured to move longitudinally within the central lumen of the elongated cylinder between a compact storage position in which the distal end of the handle is in proximity with the distal end of the cylinder, and a working position in which the distal end of the handle is away from the distal end of the cylinder, such that most of the handle's length protrudes in a proximal direction from a proximal end of the cylinder; wherein in the working position the handle is rotatable relative to the cylinder.

### FIELD OF THE INVENTION

The present invention is directed to a bandage applicator. More specifically, the invention relates to a disposable applicator used to apply roll bandages to an injured portion of a subject's body, particularly in an emergency or first-aid situation where the immediate cessation of bleeding by means of rapid bandaging is of the utmost importance. The applicator further comprises a removable element that can be used as a tensioning rod to achieve a tourniquet effect in the case of arterial hemorrhage.

### BACKGROUND OF THE INVENTION

The process of arresting bleeding at the body surface of an injured or ill subject is one in which speed is of the essence, to avoid loss of blood, surgical shock, and in some cases, even death. Most commonly, open bleeding wounds on the body surface are treated by applying fabric bandages of one sort or another.

Bandages that are generally formed from a single piece of fabric are generally used to support a medical dressing or a device such as an immobilizing splint. In addition, bandages may be used on their own to provide mechanical support for a limb, or to restrict the movement of a limb or other body part, when this is required to assist in the healing process. While bandages may be triangular in shape, for certain applications (e.g., to form temporary slings for immobilizing fractured arms), the roll bandage (i.e., an elongated fabric strip packaged in a rolled-up form) is the most commonly used type.

In most cases, when used to hold a dressing in place, the dressing (such as a gauze pad) is applied directly onto the wound or lesion being treated, and a bandage is then used to hold the dressing in place. In such cases (i.e., where the dressing and supporting bandage are separate units), it is important to ensure that the dressing does not move from its intended treatment site, during the application of the bandage. This is of particular importance in first aid or other emergency situations, where the accurate placement of dressings to wounds and the subsequent tight application of a bandage to secure said dressings in place is a matter of great urgency. In such circumstances, a single operator (paramedic or untrained bystander) may need to work alone in treating the wound.

For the most part, bandaging systems have remained unchanged for more than a century, and there is a need for an improved bandaging application device which can improve the speed of bandage application, as well as permit the optimal application of pressure and tightness to the treatment site. In addition, a need exists for a system which permits the application of bandages while minimizing or entirely preventing all unnecessary contact of the operator with the bandage, to reduce the risk of infection.

It is an object of the invention to provide a bandage application device (“bandage applicator”) that simplifies the application of a bandage to a patient, particularly if only one operator exists to apply the bandage.

It is another object of the invention to provide a bandage applicator that accelerates the application of a bandage to a patient's limb.

It is still another object of the invention to provide a bandage applicator that includes a built-in element for performing a tourniquet effect in the case of arterial hemorrhage.

It is still an object of the invention to provide said bandage applicator in a simple, lightweight, low-cost, and compact form, that enables easy packaging in a sterile form.

It is still another embodiment of the invention to provide said applicator in a disposable form, preventing any try to reuse it, and preventing possible infection due to said future reuse.

Other objects and advantages of the invention become apparent as the description proceeds.

### SUMMARY OF THE INVENTION

The invention relates to a two-state roll-bandage applicator, comprising (a) an elongated cylinder configured for insertion into a central lumen of a roll bandage, said cylinder comprising one or more flanges configured to retain the cylinder within the lumen of said roll bandage; (b) a handle configured to move longitudinally within the central lumen of the elongated cylinder between a compact storage position in which the distal end of the handle is in proximity with the distal end of the cylinder, and a working position in which the distal end of the handle is away from the distal end of the cylinder, such that most of the handle's length protrudes in a proximal direction from a proximal end of the cylinder; wherein in the working position the handle is rotatable relative to the cylinder.

In an embodiment of the invention, said handle comprising one or more flanges and said cylinder comprising one or more first internal protrusions within the cylinder's lumen, that together interact to lock the handle at said storage position.

In an embodiment of the invention, said lock of the handle at said storage position is releasable, to allow a user to transfer the applicator to its working position.

In an embodiment of the invention, said cylinder comprising one or more second internal protrusions at the cylinder's lumen, wherein said handle's flanges are configured to engage said second protrusions such that when the applicator is in said working state, a further longitudinal movement of the handle to either the proximal direction or to the distal direction is eliminated.

In an embodiment of the invention, said elimination of longitudinal movement in said working state prevents both the removal of the handle from the cylinder, and returning the handle to its storage position, thereby preventing multiple use of the applicator.

In an embodiment of the invention, a separate release element enables a longitudinal movement of the handle out of the working state, and back to the storage state or entirely out of the cylinder, thereby to enable multiple use of the applicator during training.

In an embodiment of the invention, the handle further comprising a handle's lumen configured to contain a removable central plug, said plug is suitable in size and shape for use as a tensioning rod that increases the pressure applied by the bandage on a wounded limb.

In an embodiment of the invention, the central plug has a rod section and a disk-like or dome senction, and wherein the rod section includes a thread configured to allow retain of the plug within the handle's lumen.

In an embodiment of the invention, at least one of the hangle flanges has an arrowhead shape directed to the distal direction.

The invention also relates to a bandaging assembly comprising the above bandage applicator a roll bandage, wherein the cylinder of the applicator is retained within the lumen of the roll bandage.

In an embodiment of the invention, the roll bandage is a self-adhesive roll bandage.

In an embodiment of the invention, a wound dressing material is pre-attached to one face of the roll bandage, close to its outer end.

In an embodiment of the invention, the wound dressing material comprises a medicinal composition.

In an embodiment of the invention, the medicinal composition comprising a blood-clot-promoting substance.

In an embodiment of the invention, the cross-section of lumen of the roll bandage, as well as the outer cross-section of the cylinder are not circular.

### BRIEF DESCRIPTION OF THE DRAWINGS

In the drawings:

FIGS. 1 to 3 illustrate the components of the bandage applicator of the invention;

FIGS. 4 and 5 illustrate the applicator in its storage state;

FIGS. 6 and 7 illustrate the applicator of the invention in its working state, while in FIG. 7 the central plug is shown separated from the handle;

FIGS. 8 and 9 show the distal end of the applicator while being retained within the lumen of the roll bandage;

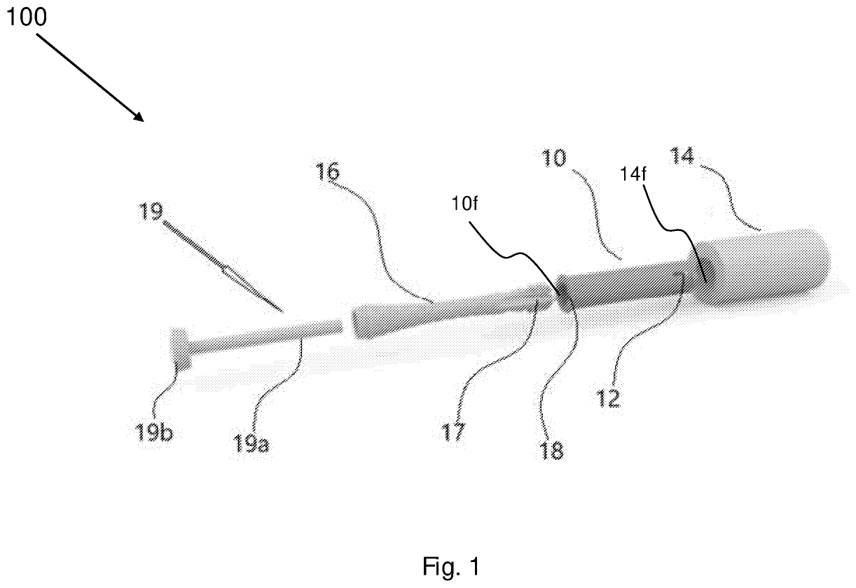

FIGS. 1 to 12 show cross-sections of the applicator in various states;

FIG. 13 shows a bandage assembly that includes the applicator of the invention attached to roll bandage; and

FIGS. 14a to 14b illustrate how the central plug of the applicator is used to tighten the bandage thereby achieving a tourniquet effect in the case of arterial hemorrhage.

### DETAILED DESCRIPTION OF PREFERRED EMBODIMENTS

The invention is primarily directed to a roll-bandage applicator, whose purpose is to facilitate the application of a roll bandage to an affected body part. The bandage is typically provided rolled around a hollowed cylindrical cardboard (or polymer) core. The bandage applicator permits easy and rapid rotation of the bandage, while applying it on the wounded limb. The applicator comprises, in its most general form, an elongated cylinder adapted for insertion into the central lumen of a roll bandage, a bandaging handle adapted to move longitudinally within a central lumen of the elongated cylinder between a state position and an open state. In the closed state, the distal end of the handle is substantially aligned with the distal end of the cylinder. In the open (“working”) state, the distal end of the handle is positioned at a location close to the proximal end of the cylinder, such that most of the length of the handle protrudes in a proximal direction from the proximal end of the cylinder. The applicator may further comprise a central plug which is configured to be contained within an inner lumen of the handle. Preferably, the central plug comprises an elongated portion fitted with an external screw thread (which, in this embodiment is designed to engage with a complementary internal screw thread in the wall surrounding the inner lumen of the handle), and an expanded disk-like (or dome like) portion at its proximal end. During assembly of the applicator in the factory, the central plug is inserted into the lumen of the handle, preferably by means of screwing it into place, such that the proximal disk-like region of the central plug protrudes from the proximal end of the handle. The use of the central plug as a bandage tensioning rod will be described hereinbelow.

It is to be noted that the term “proximal” as used herein, refers to the end of the applicator and its components which, in use are closest to the operator and furthest from the wound or lesion that is being dressed. Conversely, “distal” refers to the direction which is closest to the wound being dressed and farthest from the operator (or the operator's hand being used in self-application of the bandage, as will be describe hereinbelow).

One embodiment of the bandage applicator 100 of the invention is illustrated in FIG. 1, showing the above-defined components presented prior to their assembly into the finished product. Cylinder 10 is shown toward the right side of the figure. The cylinder includes a plurality of flanges 12 having distal elements used to lock the cylinder (as described hereinbelow) within a central lumen of roll bandage 14. To the left of cylinder 10, the figure shows the bandaging handle 16, which (in this embodiment) has a tapering outer diameter, such that the middle portion along the length thereof is narrower than either the proximal or distal ends, thereby enabling the handle to be conveniently held in the hand of the operator. In addition, in this embodiment, the outer surface of the handle is textured, to facilitate an optimal grip within the operator's hand. The distal portion of the handle 16 comprises a plurality of external flanges 17 designed to engage with a similar number of grooves 18 that are cut into the proximal portion of the inner lumen of cylinder 10 to facilitate smooth insertion and retraction of the handle in and out of cylinder 10. The embodiment shown in FIG. 1 includes four such flanges 17 and respective grooves 18, but it is also possible to construct the applicator with a smaller or larger number of these elements, respectively. Bandaging handle 16 further comprises an inner flange or tab (not shown in FIG. 1) used as part of a locking mechanism to prevent unintentional retraction of the handle to its extended, working position.

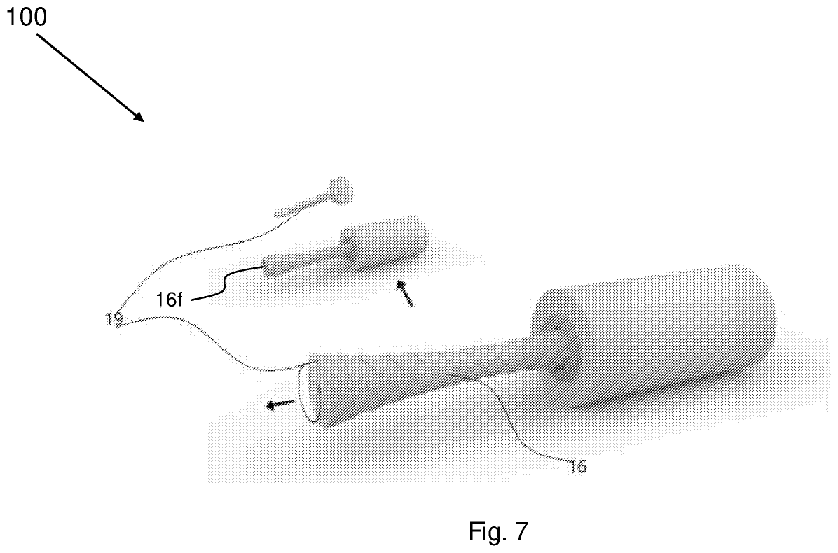

It should be noted that when handle 16 is fully inserted into lumen 10f of cylinder 10, it is locked within the cylinder, and rotation between the handle and the cylinder is prevented. However, when handle 16 is in its retracted position, as shown in FIG. 7, a rotation between handle 16 and cylinder 10 becomes possible. This feature is important when operating the bandage retractor. The above mechanism is discussed in more detail hereinbelow. Finally, the left side of FIG. 1 shows the central plug 19, comprising an elongated rod 19a and a proximally located expanded disk 19b.

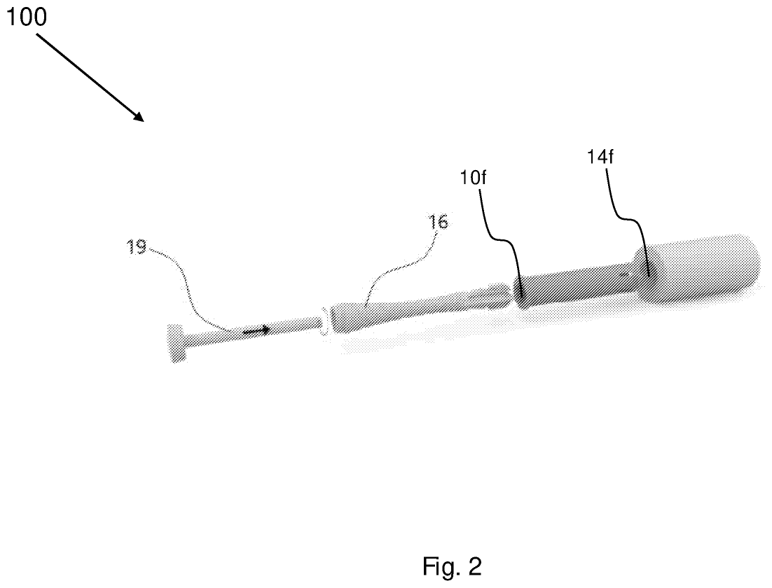

As mentioned hereinabove, in some embodiments of the invention, the applicator comprises a central plug 19 that is fitted, during product assembly, into the inner lumen 16f (FIG. 7) of the bandaging handle 16. It may be seen from FIG. 2 that the rod section of the central plug 19 is rotatably inserted into the central lumen 16f of bandaging handle 16 (in the direction of the arrow) and thus becomes an integral part of the handle during packaging and normal use. Plug 19 has an additional function, namely, to act as a bandage tensioning rod. As shown in FIGS. 14a, in some instances, the plug is inserted underneath the bandage substantially parallel to the wounded limb's surface, during or after application of the bandage, and rotation of the rod increases the bandage tightening and mechanical force applied by the bandage to the wound or lesion (such as in the case of arterial or another profuse hemorrhage). Therefore, the tightened bandage (by the rod, after its rotation) is used to arrest arterial bleeding. As noted, to use the central plug 19 as a tensioning rod, the plug is removed from the central lumen of bandaging handle 16 (in a direction opposite to that indicated by the arrow shown in FIG. 2), inserted underneath the bandage (as shown in FIG. 14a) and rotated by the user's fingers in a direction perpendicular to the longitudinal axis of rod 19a. Such plug rotation tightens the bandage and applies more pressure on the limb, as shown in FIG. 14b.

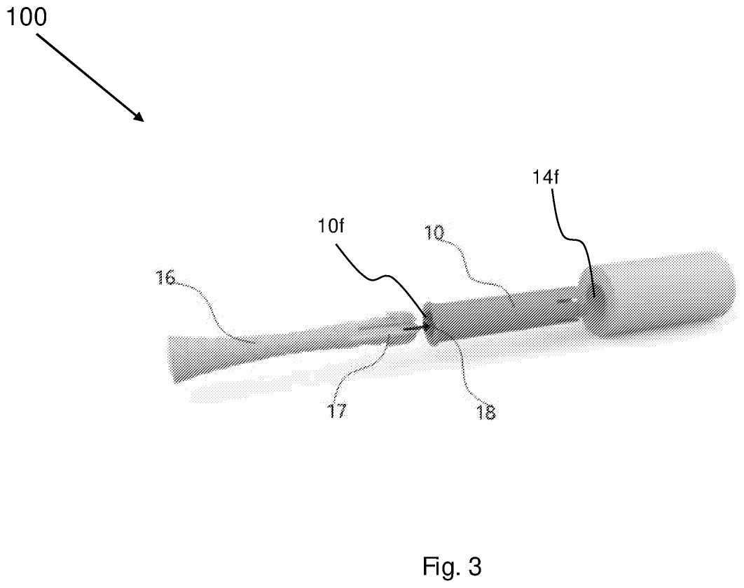

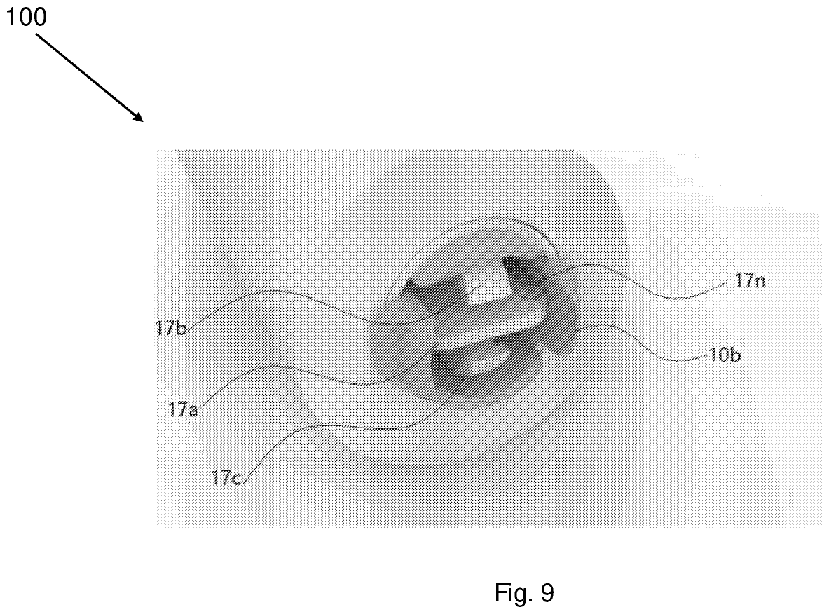

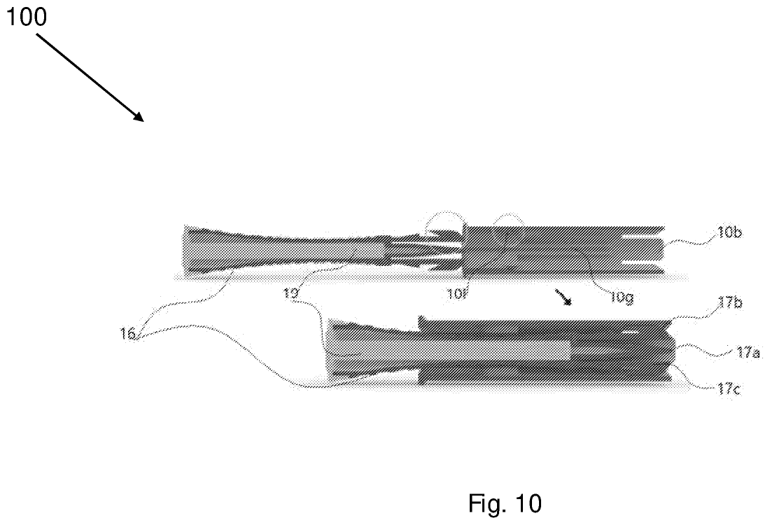

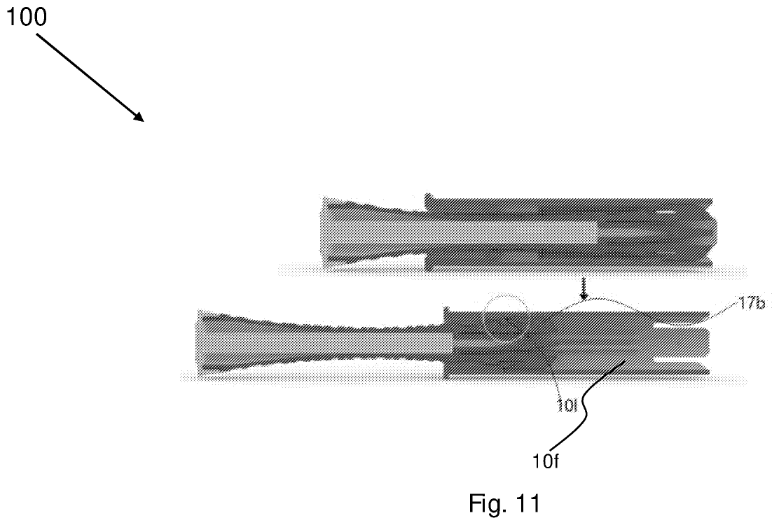

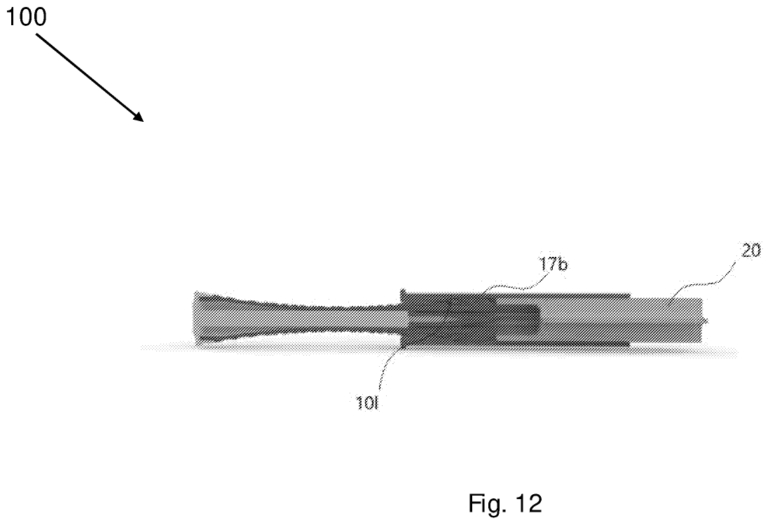

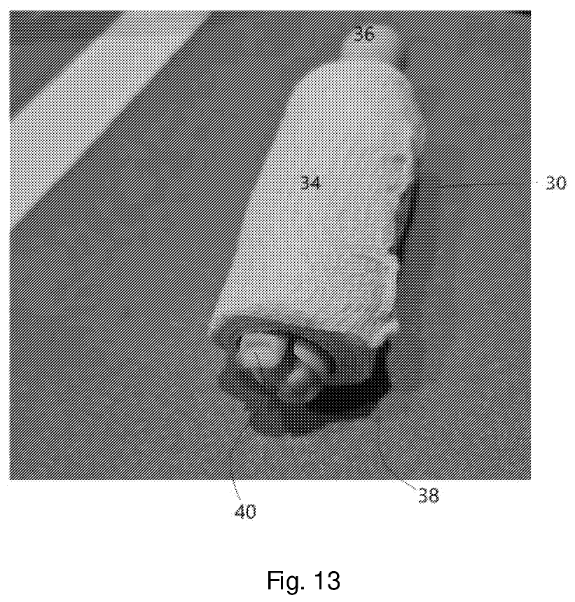

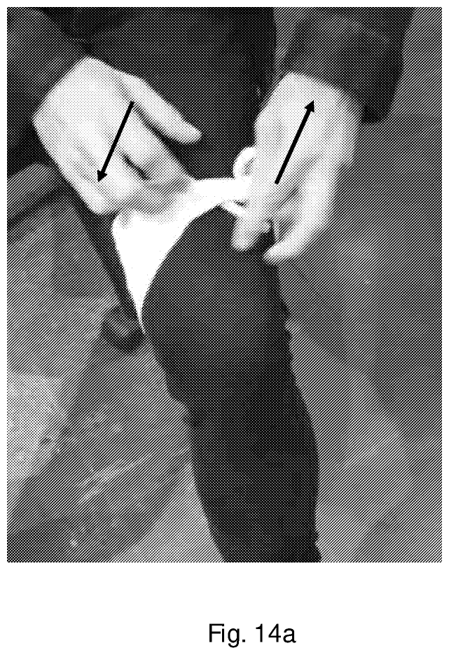

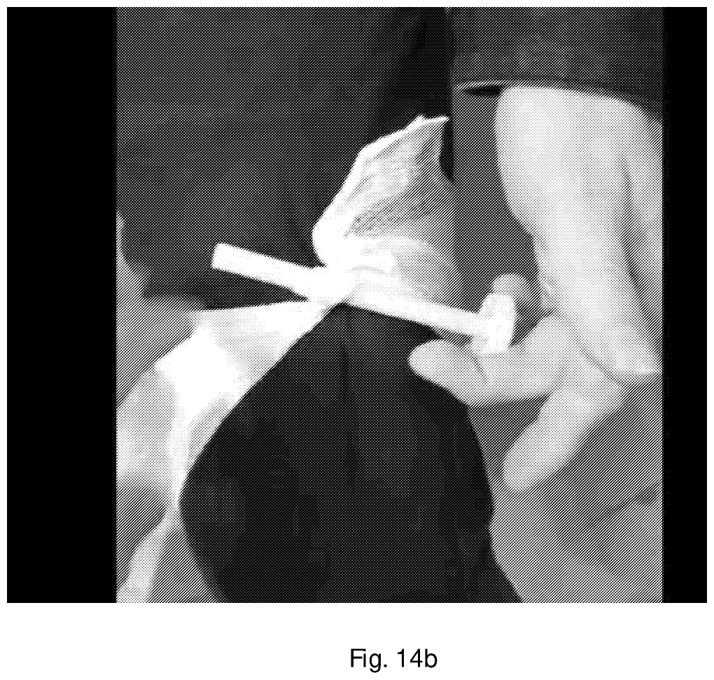

FIG. 3 illustrates the next stage of assembly of the bandage applicator of the present invention, namely the insertion of bandaging handle 16 into the central lumen of cylinder 10. As mentioned hereinabove, the rotational alignment of the handle within said cylinder is facilitated by the presence of external flanges 17 (FIG. 9) at the distal end of the handle, which engage with slots 18 (FIG. 1) in the inner wall of the cylinder.

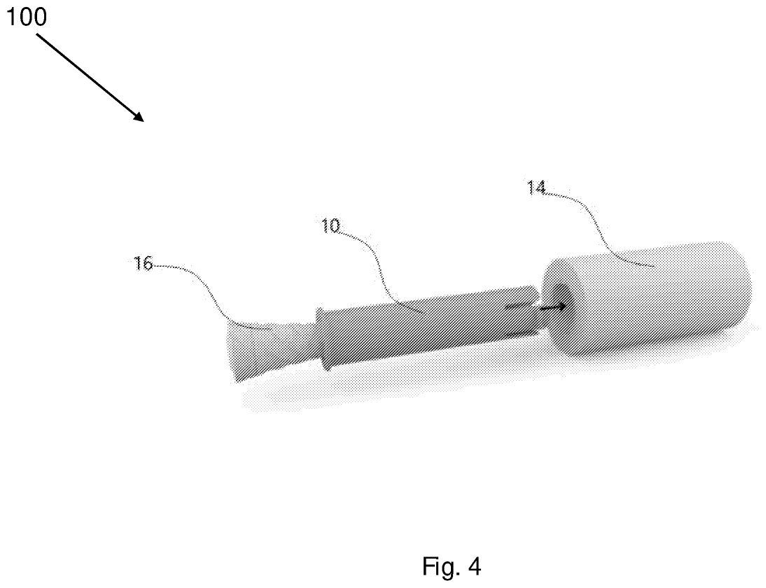

The final stage of the assembly according to this bandage applicator's embodiment is shown in FIG. 4. Thus, it is seen that the bandaging handle 16 has been fully inserted into the inner lumen of cylinder 10, such that only a small proximal terminal portion of said handle is visible, protruding beyond the proximal end of the cylinder. In this stage, the applicator has a compact size, ready for sterilized packaging and use. To use the applicator with roll bandage, cylinder 10 is inserted into the inner lumen of roll bandage 14, in the direction of the arrow, as shown.

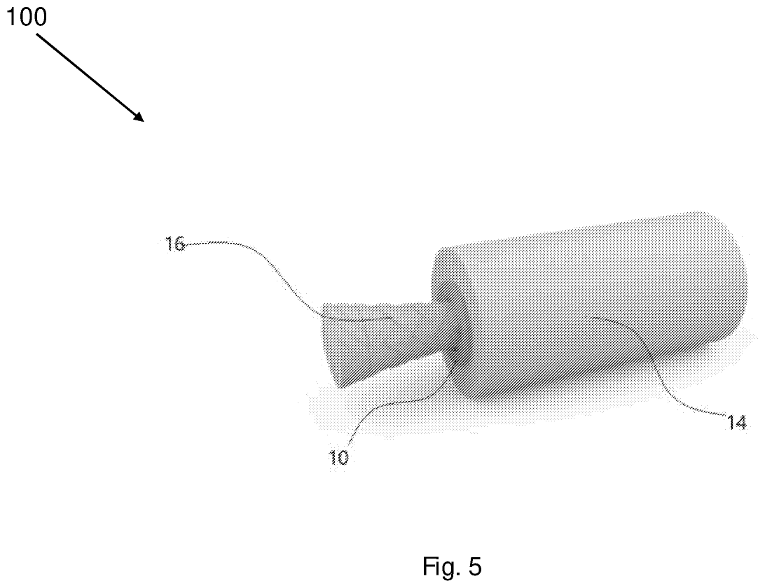

FIG. 5 depicts the applicator 100 fully assembled within the roll bandage 14. In this initial state, the bandaging handle 16 is in its closed position, i.e., only slightly protruding beyond the proximal extremity of the cylinder 10 and roll bandage 14. Following assembly, the applicator is packaged and sterilized prior to distribution, storage and use. In another embodiment, each of the applicator and roll bandage may be packaged separately (in sterilized packages), and assembled just before use.

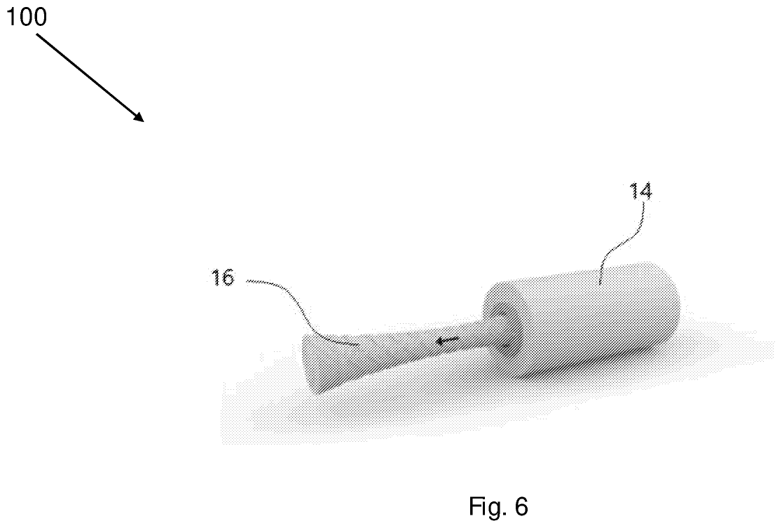

When necessary, assuming that both the roll bandage and the applicator are packaged assembled, the assembled structure is removed from the sterilized packaging. As shown in FIG. 6, the bandaging handle 16 is retracted proximally (away from roll bandage 14) within the cylinder's lumen 10f to the retracted state of the applicator, becoming locked in its fully open, working position. Utilizing this locking mechanism (discussed in more detail below), pushing the handle 16 further back inside the cylinder is impossible. This mechanism, therefore, ensures that the applicator is for single use only, and cannot be reused, such as reloaded with a new bandage and then repackaged. However,

As mentioned hereinabove, the central plug 19 is designed to be removable from its housing within the handle, such that it can be used as a tensioning rod, when it becomes necessary. FIG. 7, in its lower portion, depicts the assembled applicator 100, in its open state. To remove central plug 19 from the handle, the expanded disk-like portion thereof is grasped and rotated (as shown by the circular arrow in the figure) until the plug is removed from handle 16, as depicted in the upper part of FIG. 7.

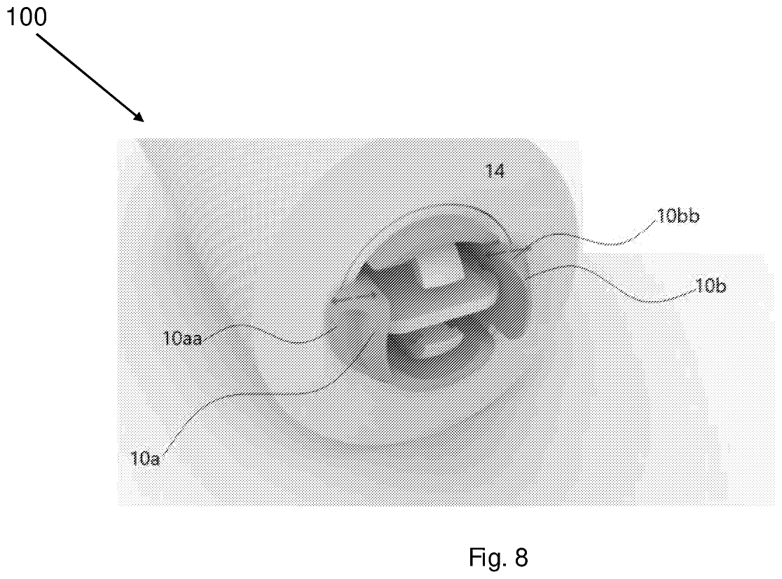

FIG. 8 provides a close-up view of the distal end of the assembled applicator. As mentioned, cylinder 10 ends in a plurality of flanges. In the embodiment shown in this figure, the applicator has four such flanges, two of which (10a and 10b) include at their distal ends an expanded tooth-like region (e.g., 10aa at the distal extremity of 10aa). Thus, during assembly of the applicator, after the cylinder has been pushed into the central lumen 14f of roll bandage 14, teeth 10aa and 10bb spring outwards (in the direction indicated by the horizontal arrows), gripping the distal face of the roll bandage 14, thus locking the bandage in place over the cylinder.

FIG. 9 illustrates an additional novel mechanism incorporated into the applicator of the invention, namely a mechanism for locking a longitudinal movement of handle 16 within the cylinder and preventing its unintentional retraction therefrom. The bandaging handle (17) is divided into a plurality of flanges in its distal portion as described hereinabove. In the case of the embodiment depicted in FIG. 9, there are three such flanges, two outer flanges 17b and 17c, and a central flange 17a. As seen in the figure (and circumscribed in a red circle), central flange 17a includes a small notch 17n on each of its lateral edges. Following insertion of the bandaging handle 16 into the cylinder (during product assembly), each of said notches engages a small tooth present on the inner face of the opposing terminal flange of the cylinder 10 (e.g., 10b on the right side of the applicator shown in this figure). This interaction between the tooth and adjacent notch provides sufficient force to retain the bandaging handle in its inner storage position, preventing unintentional withdrawal of the handle before use. However, when the operator wishes to pull the handle proximally into its fully extended operating position immediately before use, it is necessary to exert a small pulling force on the handle (in a distal-to-proximal direction) to release notch 17n from its engagement with the small tooth.

FIG. 10 is a side view of the applicator, providing a more detailed look at the process of inserting the bandaging handle 16 into the cylinder (10) while assembling applicator 100. This figure also clearly shows the position of the central plug 19 within the central lumen of handle 16. The upper part of the figure shows the applicator before insertion of the handle, and it may be seen that the cylinder (as described hereinabove) terminates distally in a series of flanges, including flange 10b (which was also depicted in FIG. 9). The lower part of FIG. 10 illustrates the applicator after bandaging handle 16 has been inserted into its locked position within the central lumen 10f of cylinder 10. As shown, the terminal portion of the handle is divided into a series of flanges, three of which are shown in the figure as 17a, 17b, and 17c. Although not seen in this view, the handle is locked in place by virtue of the interaction between the notch in flange 17a and the small notch in cylinder flange 10b. (This interaction is shown more clearly in FIG. 9, as described above.) As will be seen from this figure, the cylinder also includes lateral grooves 10g that facilitate smooth entry of central handle flange 17a during insertion of the handle into the cylinder. Also shown in this figure is locking projection 101 formed in the proximal portion of the inner wall of the cylinder. This structure prevents the handle from moving forward inside the cylinder after being retracted before use.

FIG. 11 is another side-view illustration of the applicator of the invention, with the upper part of the figure showing the position of the bandaging handle within the central lumen of the cylinder following assembly (i.e., as in the lower portion of FIG. 10). The lower part of FIG. 11 illustrates the position of the handle following its retraction into its extended, working position. Thus, it can be seen that the lateral handle flanges (including 17b) become locked into place by engaging with locking projection 101, which is formed within the proximal portion of the inner wall of the cylinder. As shown, projection 101, together with the complementary recess in flange 17b, prevents the handle from being completely withdrawn (i.e., into the pre-assembled position) or re-inserted into the cylinder to the compact position used for packaging and sterilization (however, rotation of the handle 16 relative to cylinder 10 becomes possible). In this way, the invention can be used only as a single-use, throw-away applicator. However, since there may be situations in which it is necessary or desirable to reuse the applicator—for example, to train new operators in the use of the bandaging applicator—a custom-made release element 20 shown in FIG. 12 may be used. Thus, as shown in this figure, release element 20 is inserted through the open distal end of the cylinder and advanced proximally until the distal ends thereof engage with the lateral flanges (17b) of the bandaging handle. By continuing to apply longitudinal pressure on the distal end of release element 20, the proximal ends of said release element press proximally on said lateral handle flanges. Since the distal ends of these flanges are angled, as shown in the figure, such pressure causes the lateral flanges to move inwards, towards the center of the cylinder's inner lumen, thus releasing the handle from the confines of the locking projection 101. The handle may then be pushed distally back into its pre-use storage position.

It is to be noted that the two locking mechanisms described hereinabove (i.e., the mechanism for locking the handle in its closed position, as shown in FIG. 10, and the mechanism for locking the handle in its open position, as shown in FIG. 11), are integral parts of the present invention. As described above, concerning FIGS. 10 and 11, said locking mechanism comprise a series of flanges on the outer surface of the distal portion of the handle which interact with projections formed in the inner wall of the cylinder (with the closed-position locking elements located in the distal portion of said inner wall, and the open-position locking elements located in the proximal portion thereof). It is to be further noted that while the present disclosure described these locking mechanisms in the context of a bandage applicator system, said mechanisms may also be incorporated into other applicators having rotatable sleeve portions and extendable handles included therein. Examples of such additional applicators include roller brushes for cleaning clothes, paint rollers, electrician rollers, etc.

FIG. 13 is a photograph showing an applicator of the invention 30, fully assembled and loaded with a roll bandage 34, with the handle 36 stored within the inner lumen of the cylinder 38. The handle locking mechanism 40 may be clearly seen at the distal edge of the applicator.

The applicator of the present invention possesses many advantages in comparison with prior art bandaging systems, including, but not limited to:
Effective and rapid application of the bandage to the affected site;
The use of a bandage that adheres to itself, layer-over-layer;
The applicator it self is low-cost and is designed for hygienic, single use and disposable, preventing second use.

The bandage applicator of the present invention is primarily intended for use by paramedics, emergency responders, and injured persons themselves (e.g., soldiers) in first aid and other emergencies medical treatment settings, such as in accidents, scene-of-crime, and on the battlefield. In this regard, it is also to be noted that the operation of the applicator is simple enough to permit its use by untrained personnel, and therefore it may be included among the equipment and supplies found in first-aid kits intended for domestic and institutional use, as well in cars, trains and other forms of public and private transportation. The bandage applicator is also highly suitable for use within hospitals and other clinical settings, for example in the operating theater, outpatient clinics, inpatient wards, emergency rooms, and so on. In some embodiments of the invention (particularly when the applicator is used in one-handed mode), the application process ends when the inner end of the bandage is stripped off its core. In other embodiments, the operator may choose not to use the entire length of bandage, but rather to cut the bandage at a certain length using scissors. The bandage itself may be of the adhesive type, or a regular, non-adhesive type (white-type).

The adhesive nature of the roll bandage used in the present invention obviates the absolute need for means to secure the outer end of the bandage in place (e.g., by splitting the end and tying the two free ends thus formed together, or by using a length of surgical tape or a metal clip). However, such means may also be used in conjunction with the present invention, if desired.

The bandage applicator of the present invention may be constructed from any suitable non-hazardous material, including, but not limited to polymeric materials such as polypropylene. The applicator may be constructed using any of the standard manufacturing processes well known to the skilled artisan including additive manufacture (3D printing), injection molding, and so on.

Various commercially available adhesive roll bandages may be used in conjunction with the applicator of the present invention. As mentioned hereinabove, the bandage applicator of the present invention may be used in conjunction with an adhesive roll bandage and separate wound dressing materials (e.g., gauze pads, gels, plastic films, foams, hydrogels etc.). In another embodiment, the roll bandages may also have the wound dressing material sewn (or otherwise attached) to one surface of the bandage, near to its outer end. In one embodiment, said wound dressing material may also incorporate a medicinal composition, such as an antiseptic agent (e.g., povidone) or a haemostatic agent designed to promote rapid blood coagulation (e.g., a chitosan-derived product, such as Celox). Moreover, the applicator 100 of the invention may be used with a roll bandage in which the lumen may have any cross-sectional shape (in addition to a circular cross-section, as shown in the figure). For example, the roll bandage lumen may have an elliptic cross-sectional shape, a square or rectangular cross section, etc. To adapt the device to such shapes, the outer cross-section of “cylinder” 10 should be respectively made, to conform with the cross-section of the roll bandage lumen (while the cross-section of the internal lumen of the cylinder remains circular). Therefore, the term “cylinder” used in this application encompasses also all these cases.

The overall length of the bandage applicator, with the handle fully extended in its working position will generally be in the range of 15 cm to 30 cm. The cylinder portion of the applicator may be manufactured in a variety of lengths and diameters configured to accommodate roll bandages and dressings suitable for different applications, models, and manufacturers (e.g., wider bandages and dressings for abdominal and thoracic use, and narrower versions for use on limb wounds).

The bandage applicator of the invention enables the user to roll a bandage on a wounded limb by use of one hand only, while in the prior art the user has repeatedly to move the roll bandage from one hand to the other, namely, he has to use two hands. This is an important feature of the applicator of the invention. The applicator of the invention also enables the provision of more pressure to the wounded limb, by use of the built-in and removable plug.

1. A two-state roll-bandage applicator, comprising:
an elongated cylinder configured for insertion into a central lumen of a roll bandage, said cylinder comprising one or more flanges configured to retain the cylinder within the lumen of said roll bandage;
a handle configured to move longitudinally within the central lumen of the elongated cylinder between a compact storage position in which the distal end of the handle is in proximity with the distal end of the cylinder, and a working position in which the distal end of the handle is away from the distal end of the cylinder, such that most of the handle's length protrudes in a proximal direction from a proximal end of the cylinder;
and wherein in the working position, the handle is rotatable relative to the cylinder.

2. The roll-bandage applicator of claim 1, wherein said handle comprising one or more flanges and said cylinder comprising one or more first internal protrusions within the cylinder's lumen, that together interact to lock the handle at said storage position.

3. The roll-bandage applicator of claim 2, wherein said lock of the handle at said storage position is releasable, to allow a user to transfer the applicator to its working position.

4. The roll-bandage applicator of claim 1, wherein said cylinder comprising one or more second internal protrusions at the cylinder's lumen, wherein said handle's flanges are configured to engage said second protrusions such that when the applicator is in said working state, a further longitudinal movement of the handle to either the proximal direction or to the distal direction is eliminated.

5. The roll-bandage applicator of claim 1, wherein said elimination of longitudinal movement in said working state prevents both the removal of the handle from the cylinder, and returning the handle to its storage position, thereby preventing multiple use of the applicator.

6. The roll-bandage applicator of claim 5, wherein a separate release element enables a longitudinal movement of the handle out of the working state, and back to the storage state or entirely out of the cylinder, thereby enabling multiple use of the applicator during training.

7. The roll-bandage applicator of claim 1, wherein the handle further comprising a handle's lumen configured to contain a removable central plug, said plug is suitable in size and shape for use as a tensioning rod that increases the pressure applied by the bandage on a wounded limb.

8. The roll-bandage applicator of claim 1, wherein the central plug has a rod section and a disk-like or dome section, and wherein the rod section includes a thread configured to allow retention of the plug within the handle's lumen.

9. The roll-bandage applicator of claim 1, wherein at least one of the hangle flanges has an arrowhead shape directed to the distal direction.

10. A bandaging assembly comprising the bandage applicator of claim 1 and a roll bandage, wherein the cylinder of the applicator is retained within the lumen of the roll bandage.

11. The bandaging assembly of claim 10, wherein the roll bandage is a self-adhesive roll bandage.

12. The bandaging assembly of claim 10, wherein a wound dressing material is pre-attached to one face of the roll bandage, close to its outer end.

13. The bandaging assembly of claim 12, wherein the wound dressing material comprises a medicinal composition.

14. The bandaging assembly of claim 13, wherein the medicinal composition comprising a blood-clot promoting substance.

15. The roll-bandage applicator of claim 1, wherein the cross-section of the lumen of the roll bandage, as well as the outer cross section of the cylinder are not circular.
